(19) United States
(12) Patent Application Publication
(10) Pub. No.: US 2008/0007692 A1
(43) Pub. Date: Jan. 10, 2008

(54) Apparatus and method for spectrally measuring fundus

(75) Inventors: Toshifumi Mihashi, Tokyo (JP); Yoko Hirohara, Tokyo (JP); Hiroyuki Aoki, Tokyo (JP); Yasuko Tsuruga, Tokyo (JP)

Correspondence Address: FOLEY AND LARDNER LLP;SUITE 500, 3000 K STREET NW, WASHINGTON, DC, 20007, US

(73) Assignee: TOPCON CORPORATION

(21) Appl. No.: 11/812,081

(22) Filed: Jun. 14, 2007

(30) Foreign Application Priority Data
Jun. 15, 2006 (JP) 2006-166690

(51) Int. Cl. A61B 3/14

(52) U.S. Cl. 351/206

(57) ABSTRACT

To provide a spectroscopic fundus measuring apparatus capable of identifying each part in spectral fundus images easily and accurately based on its spectral characteristic and a measuring method therefor. A spectral fundus image measuring apparatus 1 of the present invention includes: an illumination optical system 10 having an illumination light source 11 for illuminating a fundus; a light receiving optical system 20 for receiving a wavelength-tunable light beam reflected from the illuminated fundus to photograph a series of spectral fundus images of different wavelengths; an image processing section 7 for processing the spectral fundus images; a storage section 7A for storing the spectral fundus images; and a display section 7B for displaying the spectral fundus images. The image processing section 7 has a position correcting section 72 for correcting the series of spectral fundus images photographed by the light receiving optical system 20 to match the positions of the same parts therein, and an image extracting section 74 for extracting spectral fundus images in wavelength ranges predetermined for respective specific parts from the series of spectral fundus images corrected in the position correcting section 72.

### BACKGROUND OF THE INVENTION

1. Technical Field

The present invention relates to a spectroscopic fundus measuring apparatus and a measuring method therefor. In particular, the present invention relates to a spectroscopic fundus measuring apparatus that acquires spectral fundus images to facilitate identification of parts with different spectral characteristics and that can form an image in which every part is clear in consideration of the spectral characteristics thereof, and a measuring method therefor.

2. Related Art

Fundus observation is doubtless important in ophthalmic diagnosis. At present, anomaly findings are obtained by diagnosing the eye fundus by means of colored fundus images, fluorescent contrast images, etc. from a fundus camera. If it is possible to measure quantitatively oxygen saturation degree on the fundus and constituent substances distributed in the retina, there is a possibility of finding out the functions of fine parts of the retina, which is considered to be greatly useful in clinical applications. Further, if spectral distribution of substances in the retina is clarified by spectral analyses, there is a possibility of analyzing the substances in the retina from the spectral images.

However, most of the studies carried out up to now are far from spectral image measurement in full-scale. Full-scale image measurement is considered to meet such conditions as: (a) being capable of obtaining high quality images, and (b) being capable of measuring spectral images with a higher degree of wavelength analysis over a wide wavelength band. Such an image measurement method is occasionally called hyper-spectral imaging. Advent of the liquid crystal wavelength tunable filter has made it possible to obtain spectral images relatively easily. Using a number of spectral images of different wavelengths makes it possible to examine spectral characteristics of substances in detail and to extract constituents having various known spectral distributions.

As a technique for identifying each part in a fundus image, a method for distinguishing retinal arteries and retinal veins based on red and green components in a fundus image has been disclosed. Also, a technique where the light beam reflected from a fundus is divided by wavelength into light beams within at least two wavelength regions in a wavelength range of 600 nm or more by a wavelength dividing means, fundus images of different wavelengths are photographed separately by a fundus photographing means, and a plurality of fundus images of different colors are superimposed to display one color image by a displaying means has been disclosed (see Patent Documents 1 and 2).

[Patent Document 1] JP-A-2001-145604 (paragraphs 0024 to 0091, FIG. 1 to FIG. 20)

[Patent Document 2] JP-A-Hei 8-71045 (paragraphs 0011 to 0032, FIG. 1 to FIG. 13)

While the hyper-spectral imaging is a technique in the spotlight and is used to obtain spectral images of the fundus, it is hard to perform accurate analyses because the amount of light of spectral images obtained varies greatly by the wavelength. Moreover, the hyper-spectral light separation with a light amount without putting burden on humans has yet to be realized.

A liquid crystal wavelength tunable filter, which was recently developed, is commercially available and can be used in spectroscopic imaging. When it is used, hyperspectral imaging of a retina can be easily achieved. However, as restricted by for example the wavelength tunable time of the liquid crystal wavelength tunable filter and the exposure time of the camera, it takes about 20 seconds to take images at every 10 nm in the wavelength range from 500 nm to 720 nm. Because alignment between the eye and the apparatus varies during that time, there has been another problem that the spectral images taken of the same part are displaced from each other.

The present inventors proposed an apparatus and method for measuring spectral fundus image data that can eliminate position displacement between spectral images of the same part even if change in alignment occurs between the eye and the apparatus with the lapse of time in Japanese Patent Application No. 2004-352093. However, there is still desired a spectroscopic fundus measuring apparatus capable of identifying parts with different spectral characteristics in a spectral fundus image easily and accurately and forming an image in which every part is clear and a method therefor.

An object of the present invention is to provide a spectroscopic fundus measuring apparatus capable of identifying each part in spectral fundus images easily and accurately based on its spectral characteristic and a measuring method therefor.

### SUMMARY OF THE INVENTION

To solve the above mentioned problem, a spectroscopic fundus measuring apparatus related to aspect (1) of the present invention 1 comprises, as shown in FIG. 1 for example, an illumination optical system 10 having an illumination light source for illuminating a fundus; a light receiving optical system 20 for receiving a wavelength-tunable light beam reflected from the illuminated fundus to photograph a series of spectral fundus images of different wavelengths; an image processing section 7 for processing the spectral fundus images; and a storage section 7A for storing the spectral fundus images, the image processing section 7 having a position correcting section 72 for correcting the series of spectral fundus images photographed by the light receiving optical system 20 to match the positions of the same parts therein, and an image extracting section 74 for extracting spectral fundus images in wavelength ranges predetermined for respective specific parts from the series of spectral fundus images corrected in the position correcting section 72.

Here, a series of spectral fundus images of different wavelengths typically mean a group of spectral fundus images of the same subject eye photographed in succession while increasing or decreasing the wavelength in a predetermined wavelength range. Changes in the photographing order or slight changes in the photographing conditions are acceptable. Processing means image processing including position matching correction among spectral fundus images, transformation such as projective transformation, filtering such as noise removal and edge detection, and expanding and thinning a line. The predetermined wavelength ranges typically mean ranges in which relatively clear images of respective specific parts can be obtained. The ranges may be determined by measuring the contrasts between the specific parts and their backgrounds or according to an empirical rule. The specific parts mean distinctive parts in a fundus image such as retinal arteries, retinal veins, optic nerve head, choroid, and macula area. With the above constitution, there can be provided a spectroscopic fundus measuring apparatus capable of identifying each part in spectral fundus images easily and accurately based on its spectral characteristic.

The invention related to aspect (2) of the present invention is the spectroscopic fundus measuring apparatus related to aspect (1) wherein the image processing section 7 has an image choosing section 75 for choosing one of the extracted spectral fundus images or one of images of each of divided areas in the extracted spectral fundus images for each of the specific parts as the clearest image.

With the above constitution, a group of clear images can be preliminarily extracted for each specific part and the clearest image is chosen therefrom. Thus, the clearest image can be chosen efficiently and the choice of an improper image can be avoided.

The invention related to aspect (3) is the spectroscopic fundus measuring apparatus related to aspect (2) wherein the image choosing section 75 chooses the image in which the contrast between the brightness of a specific part and the brightness of the background thereof is the highest from the extracted spectral fundus images as the clearest image for the specific part.

Here, although the contrast between the brightness of each specific part and the brightness of its background is preferably expressed as the difference between the brightness of the specific part itself and the brightness of a background adjacent to the specific part, it may be expressed using the maximum brightness and the minimum brightness (one of them can be regarded as the brightness of the specific part and the other as the brightness of its background) in the area. With this constitution, the choice of the clearest images can be made automatically by calculating to obtain the contrasts from the series of spectral fundus images and comparing them.

The invention related to aspect (4) is the spectroscopic fundus measuring apparatus related to aspect (2), further comprising: a display section 7B for displaying the extracted spectral fundus images, wherein the image choosing section 75 chooses an image designated by an operator for a specific part from the spectral fundus images extracted and displayed on the display section 7B as the clearest image for the specific part.

With this constitution, the clearest image can be confirmed by human eyes before being chosen.

The invention related to aspect (5) is the spectroscopic fundus measuring apparatus related to any one of aspects (2) to (4), as shown in FIG. 1 for example, wherein, in the case where the image choosing section 75 chooses one of images of each of divided areas as the clearest image, the image processing section 7 has an image connecting section 76 for connecting the clearest images of all the areas to form the clearest images of the entire fundus.

With this constitution, a synthesized image in which the specific parts are visible more clearly than in the photographed images can be obtained.

The invention related to aspect (6) is the spectroscopic fundus measuring apparatus related to aspect (4), as shown in FIG. 1 for example, wherein the image processing section 7 has an image analyzing section 73 for calculating a spectral characteristic of each part on the spectral fundus images based on the series of spectral fundus images corrected in the position correcting section 72, wherein the storage section 7A stores the spectral characteristics of the parts together with standard spectral characteristics of the specific parts, and wherein, when the operator designates a part on the spectral fundus images, the display section 7B displays the spectral characteristic of the designated part stored in the storage section 7A.

With this constitution, the specific parts can be extracted with reference to the data with high identifiability.

The invention related to aspect (7) is the spectroscopic fundus measuring apparatus related to aspect (4) or (6), wherein the image analyzing section 73 calculates the contrast between the brightness of each of the parts and the brightness of the background thereof based on the series of spectral fundus images corrected in the position correcting section 72, wherein the storage section 7A stores the contrasts of the parts, and wherein, when the operator designates a part on the spectral fundus images, the display section 7B displays the contrast of the designated part stored in the storage section 7A.

With this constitution, the specific parts can be extracted with reference to the data.

The invention related to aspect (8) is the spectroscopic fundus measuring apparatus related to any one of aspects (3) to (7), wherein the image processing section 7 has an image synthesizing section 78 for synthesizing the clearest images of the entire fundus chosen by the image choosing section 75 or the clearest images of the entire fundus formed by the image connecting section 76 to form a synthesized fundus image.

With this constitution, a fundus image in which a plurality of specific parts are clearly visible can be formed.

The invention related to aspect (9) is the spectroscopic fundus measuring apparatus related to aspect (6), wherein the image processing section 7 compares spectral fundus images of a wavelength of around 580 nm and spectral fundus images of a wavelength of around 550 nm in the series of spectral fundus images and determines higher-brightness parts in the former than in the latter as retinal arteries and higher-brightness parts in the latter than in the former as retinal veins.

With this constitution, the retinal arteries and the retinal veins can be distinguished efficiently.

The invention related to aspect (10) is the spectroscopic fundus measuring apparatus related to any one of aspects (1) to (9), wherein the wavelength of the illumination light beam from the illumination light source 11 is tunable or the illumination optical system 10 or the light receiving optical system 20 has a wavelength selective filter 32. Here, when the illumination optical system 10 has a wavelength selective filter 32, the wavelength selective filter 32 is used instead of, for example, the spectral characteristic correcting filter 13. With this constitution, a series of photographed images can be obtained at wavelength intervals of, for example, 10 nm.

To solve the above mentioned problem, a spectroscopic fundus measuring method related to aspect (11) of the present invention, as shown in FIG. 4 for example, comprises: a subject eye illuminating step (S001) of illuminating a fundus of a subject eye of an animal with a light beam from an illumination light source 11 emitting a light beam in a specified wavelength range; a photographing step (S002) of receiving a wavelength-tunable light beam reflected from the illuminated fundus to photograph a series of spectral images of the fundus of the animal of different wavelengths; an image processing step of processing the spectral fundus images; and a storing step (S004) of storing the spectral fundus images, the image processing step having a position correcting step (S003) of correcting the series of spectral fundus images photographed in the light receiving optical system 20 to match the positions of the same parts therein, and an image extracting step (S005) of extracting spectral fundus images in wavelength ranges predetermined for respective specific parts from the series of spectral fundus images corrected in the position correcting step. With the above constitution, there can be provided a spectroscopic fundus measuring method capable of identifying each part in spectral fundus images easily and accurately based on its spectral characteristic. Here, the animal includes humans and living creatures other than humans.

The invention related to aspect (12) is the spectroscopic fundus measuring method related to aspect (11), as shown in FIG. 4 for example, wherein the image processing step has an image choosing step (S007) of choosing one of the extracted spectral fundus images or one of images of each of divided areas in the extracted spectral fundus images for each of the specific part as the clearest image, and, in the case where one of images of each of divided areas is chosen as the clearest image in the image choosing step, an image connecting step (S008) of connecting the clearest images of all the areas to form the clearest images of the entire fundus. With the above constitution, a group of clear images can be preliminarily extracted for each specific part and the clearest image is chosen therefrom. Thus, the clearest image can be chosen efficiently and the choice of an improper image can be avoided.

According to the present invention, there can be provided a spectroscopic fundus measuring apparatus capable of identifying each part in spectral fundus images based on its spectral characteristic easily and accurately and a measuring method therefor.

This application is based on the Patent Applications No. 2006-166690 filed on Jun. 15, 2006 in Japan, the contents of which are hereby incorporated in its entirety by reference into the present application, as part thereof.

The present invention will become more fully understood from the detailed description given hereinbelow. However, the detailed description and the specific embodiment are illustrated of desired embodiments of the present invention and are described only for the purpose of explanation. Various changes and modifications will be apparent to those ordinary skilled in the art on the basis of the detailed description.

The applicant has no intention to give to public any disclosed embodiment. Among the disclosed changes and modifications, those which may not literally fall within the scope of the patent claims constitute, therefore, a part of the present invention in the sense of doctrine of equivalents.

The use of the terms “a” and “an” and “the” and similar referents in the context of describing the invention (especially in the context of the following claims) are to be construed to cover both the singular and the plural, unless otherwise indicated herein or clearly contradicted by context. The use of any and all examples, or exemplary language (e.g., “such as”) provided herein, is intended merely to better illuminate the invention and does not pose a limitation on the scope of the invention unless otherwise claimed.

### BRIEF DESCRIPTION OF THE DRAWINGS

FIG. 1 shows an example constitution of a spectral fundus image measuring apparatus of a first embodiment.

FIG. 2 shows an example of band-pass characteristic of a liquid crystal wavelength tunable filter.

FIG. 3A and FIG. 3B show an example of a method for choosing the wavelength of the liquid crystal wavelength tunable filter.

FIG. 4 shows an example flow of a spectral fundus image data measuring method of a first embodiment.

FIG. 5 shows an example flow of matching spectral retinal image positions.

FIG. 6 shows an example flow of image position matching.

FIG. 7 shows an example of a hyperspectral image of spectral fundus images.

FIG. 8 shows an example of spectral characteristics of specific parts.

FIG. 9 shows an example of an extracted image of retinal arteries and veins and optic nerve head.

FIG. 10 shows an example flow of processing.

FIG. 11 shows examples of images during the processing.

FIG. 12A and FIG. 12B show an example of absorbed light amounts of oxygenated hemoglobin and reduced hemoglobin.

### DETAILED DESCRIPTION OF THE PREFERRED EMBODIMENT

Embodiments of the present invention are described below in reference to the drawings.

### First Embodiment

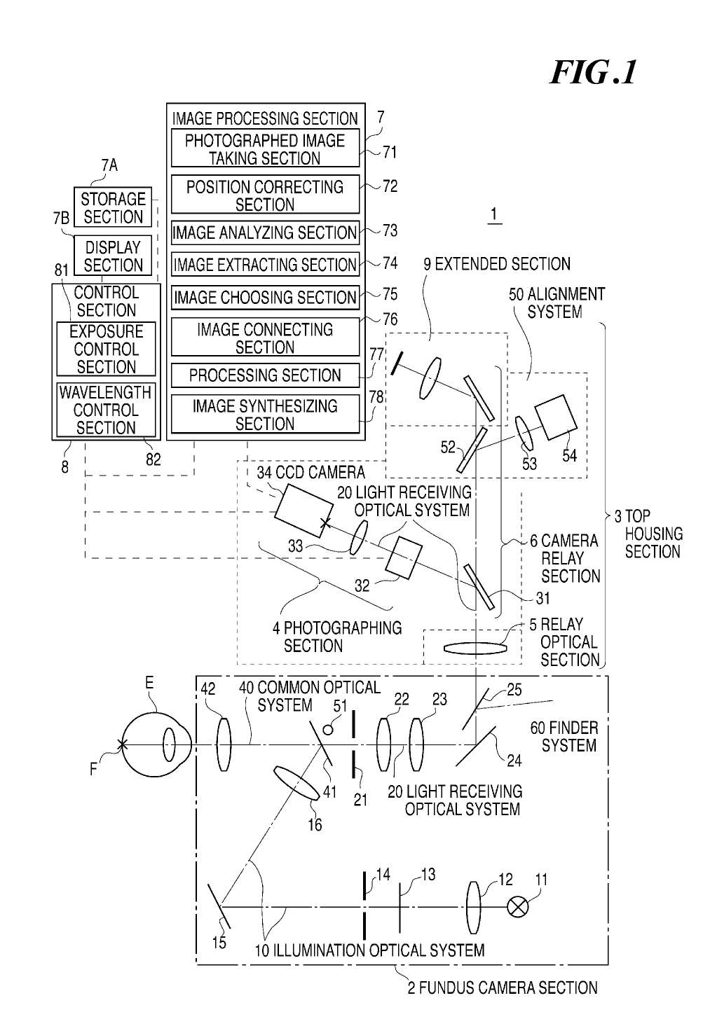

FIG. 1 shows a general example of an optical system of a spectral fundus image data measuring apparatus 1 as an embodiment of the invention. In the drawing, the spectral fundus image data measuring apparatus 1 may be roughly divided into: a fundus camera section 2, a top housing section 3, an image processing section 7, a storage section 7A, a display section 7B, and a control section 8. The fundus camera section 2 comprises: an illumination optical system 10 for illuminating the fundus F of a subject eye E, the fore stage section of a light receiving optical system 20 for receiving light beam reflected from the fundus F and forming a fundus image on the light receiving surface of a photographing section 4, a finder optical system 60 for an optometrist to observe the fundus F, etc. The top housing section 3 is made up of: the photographing section 4 for photographing a spectral fundus image, an alignment optical system 50 for aligning the illumination position of the illumination light on the fundus F (a light source 51 is provided at the fundus camera section 2), a relay optical section 5 for collimating the reflected light beam received from the fundus camera section 2 and leading it to a camera relay section 6, and the camera relay section 6 for transmitting the reflected light beam having passed through the relay optical section 5 to various light receiving means such as the photographing section 4 and comprises a hinder stage section of the light receiving optical system 20. The hinder stage section of the light receiving optical system 20 is made up of the relay optical section 5, the camera relay section 6, and the photographing section 4. An extended section 9 above the camera relay section 6 is a section for extended use by connecting various light receiving means such as a monitor TV, a hard copier, etc. to the light receiving optical system 20.

In the fundus camera section 2, the illumination optical system 10 is made up by disposing successively on its illumination optical axis: a halogen lamp 11 as an illumination light source, a condenser lens 12, a spectral characteristic correcting filter 13, a diaphragm 14, a mirror 15, a relay lens 16, and a beam splitter 41. Here, the halogen lamp 11 is placed near the front focal point of the condenser lens 12 and emits a wide wavelength range of light beam. The diaphragm 14 is disposed in a position to be conjugate with respect to the beam splitter 41.

The illumination optical system 10 further leads the light beam reflected from the beam splitter 41 through an objective lens 42 to illuminate the fundus F of the subject eye E. The area from the beam splitter 41 to the subject eye E constitutes an optical system 40 common to the illumination optical system 10 and light receiving optical system 20.

The light receiving optical system 20 is made up by disposing successively on the reflected light optical axis passing through the subject eye E: the objective lens 42, the beam splitter 41, an iris diaphragm 21, a focusing lens 22, an image forming lens 23, a mirror 24, a switching mirror 25; and is connected to the light receiving optical system of the top housing section 3. The iris diaphragm 21 is disposed in a position to be conjugate with the fore-end part of the subject eye E. When spectral images are to be taken, the switching mirror 25 is removed from the optical path, with for example a solenoid.

The alignment optical system 50 is to align the illumination light with the illuminated position on the fundus F, and is made up of a dichroic mirror 52, an image forming lens 53, and a monitoring camera 54, to observe reflected light when light is cast from the alignment light source 51 (provided in the fundus camera section 2) to the eye. The wavelength of the alignment light source 51 is set to be near infrared (for example 940 nm) so that alignment may be carried out without affecting the spectral images in the visible light range even when spectral images are being taken. The dichroic mirror 52 allows visible light (for example 750 nm or shorter in wavelength) to pass through and reflects light of longer wavelengths. The monitoring camera 54 may be for example a CCD camera. The finder optical system 60 is for an optometrist to observe the fundus F with the unaided eye. When the dichroic mirror 31 is used as a switching mirror and removed with for example a solenoid when spectral images are not being taken, it is possible to observe the fundus in color with the extended section.

In the top housing section 3, the light receiving optical system 20 has the relay optical system 5 placed on the axis of light reflected from the subject eye E, so that the light beam reflected from the fundus F is led through the relay optical system 5 into the camera relay section 6. In the camera relay section 6, a dichroic mirror 31 is placed on the reflected light axis to reflect visible light (for example 750 nm or shorter in wavelength) and allows light on the longer wavelength side to pass through. The light beam reflected from the dichroic mirror 31 is led to the photographing section 4. In the photographing section 4 are placed on the axis of light reflected from the dichroic mirror 31: a liquid crystal wavelength tunable filter 32, an image forming lens 33, and a CCD camera 34 having a light receiving surface. The light receiving surface is disposed to be conjugate with respect to the fundus F of the subject eye E, so that the fundus images are formed on the light receiving surface. The image forming lens 33 is to relay the light coming out of the liquid crystal wavelength tunable filter 32 to the CCD camera. Using the liquid crystal wavelength tunable filter 32 makes it possible to easily choose any wavelength in the visible light range and so facilitate analysis of the spectral characteristics. With this constitution, the light receiving optical system can receive a wavelength-tunable light beam reflected from the illuminated fundus to photograph a series of spectral fundus images of different wavelengths.

The image processing section 7 has a photographed image taking section 71 for taking a series of spectral fundus images photographed by the CCD camera 34; a position correcting section 72 for correcting the series of spectral fundus images to match the positions of the same parts therein; an image analyzing section 73 for calculating the spectral characteristic of each part in the spectral fundus images and the contrast between the brightness of each part in the spectral fundus images and the brightness of its background based on the series of spectral fundus images corrected in the position correcting section 72; an image extracting section 74 for extracting spectral fundus images within wavelength ranges predetermined for respective specific parts from the series of corrected spectral fundus images; an image choosing section 75 for choosing one of the spectral fundus images extracted in the image extracting section 74 or one of images of each of divided areas in the extracted spectral fundus images for each of the specific parts as the clearest image; an image connecting section 76 for connecting the clearest images of all the areas to form the clearest images of the entire fundus in the case where the image choosing section 75 chooses one of images of each of divided areas as the clearest image; a processing section 77 for performing processing such as filtering, labeling, expanding and thinning a line on the fundus images; and an image synthesizing section 78 for synthesizing the clearest images chosen for respective specific parts in the image choosing section 75 or the clearest fundus images formed for respective specific parts in the image connecting section 76 to form a synthesized fundus image, and stores programs for image position matching correction flow, spectral fundus image extracting and choosing flow, fundus image analysis flow, clearest image connecting flow, and image processing flow and so on.

The storage section 7A stores the series of spectral fundus images photographed and corrected, various fundus images such as connected, synthesized and processed fundus images, spectral characteristics and contrasts calculated based on the series of spectral fundus images, and so on. The display section 7B displays on a screen the above various fundus images, spectral characteristics, contrasts and so on.

The control section 8 controls the entire spectral fundus image measuring apparatus 1, where the objects to be controlled include actions of the fundus camera section 2, the top housing section 3, the image processing section 7, the storage section 7A, and the display section 7B, and the flow of data and signals, in order to measure spectral fundus image data. It also has an exposure control section 81 for controlling the exposure of the CCD camera 34 and a wavelength control section 82 for controlling the transmission wavelength and so on of the liquid crystal wavelength tunable filter 32, and stores programs for the flow of taking spectral fundus images and the flow of setting exposure time for the CCD camera. Incidentally, the image processing section 7 and the control section 8 may be embodied with an ordinary personal computer.

Next is described the spectral characteristic of the optical system of the spectroscopic fundus measuring apparatus of the embodiment. For the analysis of the spectral characteristics, mainly a wavelength range of 430 to 950 nm is used, within which as uniform a spectral characteristic as possible is preferable. The received light intensity can be adjusted to be within the dynamic range of the CCD camera 34 with the CCD camera 34, the liquid crystal wavelength tunable filter 32, the correction filter 13, and the halogen lamp 11 (see Japanese Patent Application 2004-352093). In this embodiment, a dispersion-type light separating method is employed as a light separation method. While the Fourier-type light separation method can be named as one other than the dispersion-type light separation method, the dispersion-type light separation method is employed because of concern about noise on the images of a retina with the Fourier-type light separation method that uses interference. Incidentally, the Fourier light separation method may alternatively be used because it can separate light instantaneously and may be sometimes advantageous in terms of the amount of light. The reasons for using a halogen lamp as the illumination light source 11 are that it emits light over a wide range of wavelength from visible light to near infrared rays, that continuous lighting for about 10 seconds is required to separate light in time sequence, and that improvement on CCDs has made it possible to take images without using a flash.

The CCD camera 34 has sensitivity over a wide range of wavelength from visible light to near infrared range, and is capable of obtaining high-definition images for example of 1,300,000 pixels (1344×1024) and of reading at a high speed (about 8 frames/sec) with low noise. The exposure control section 81 of the control section 8 adjusts the exposure time of the CCD camera 34 in order to keep the light amount received to photograph CCD images constant. When the contrast of the CCD photographed images is not sufficient, the contrast may be improved by increasing the illumination light amount of the fundus illumination light source 11 or increasing the exposure time of the CCD camera 34.

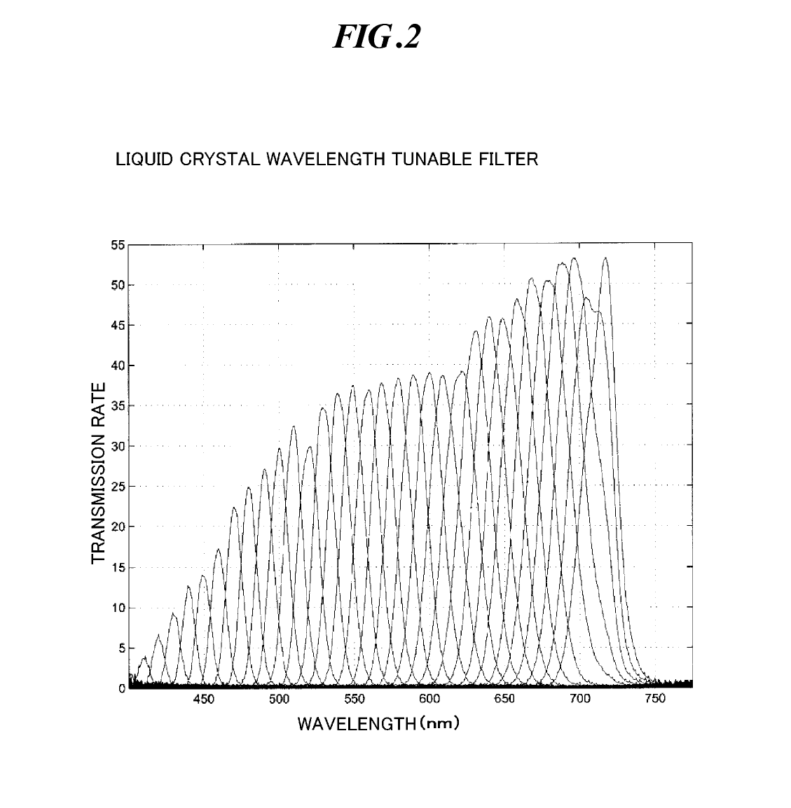

FIG. 2 shows an example of band-pass characteristic of the liquid crystal wavelength tunable filter 32. The horizontal axis represents wavelength (nm) and the vertical axis transmission rate (%). As for the liquid crystal wavelength tunable filter 32, its transmission wavelength may be chosen in the range from 400 to 720 nm by changing the voltage applied to the liquid crystal. The drawing shows how the transmission light changes when the transmission center wavelength is changed at 10 nm intervals. The wavelength width of the transmission light is about 20 nm. The peak value of the transmission light increases approximately monotonically with the increase in the wavelength.

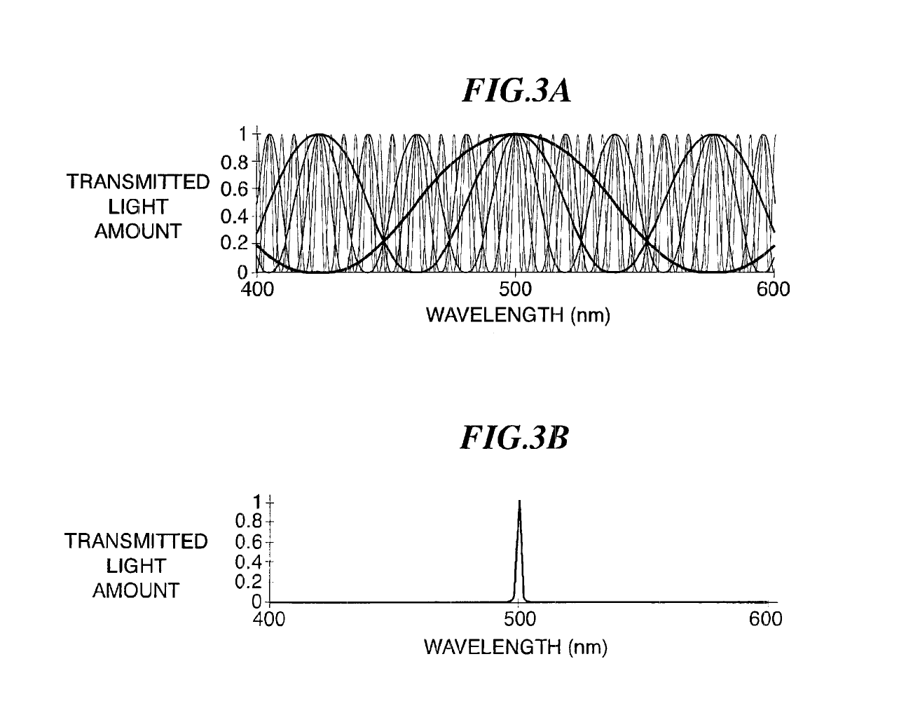

FIG. 3A and FIG. 3B show an example of wavelength choosing method with the liquid crystal wavelength tunable filter 32. Wavelength plates of different thicknesses are combined to narrow the output wavelength width, and the combinations are stacked in several stages (six stages for the example shown) to realize a wavelength width of 20 nm. FIG. 3A shows the filter characteristic of each of the Liquid Crystal Tunable Filters (LCTF) superposed in six stages. FIG. 3B shows the filter characteristic of the liquid crystal wavelength tunable filter 32 made by superposing six stages of the LCTFs. In both of the drawings, the horizontal axis represents wavelength (nm), and the vertical axis transmission rate. The transmission center wavelength may be arbitrarily changed quickly by changing the voltage applied to each LCTF, so that light of any intended wavelength component may be extracted.

Incidentally, since the liquid crystal wavelength tunable filter 32 is affected with the direction of polarization of the incident light, alignment appropriate for the polarization angle of the incident light is required when polarized light is used. In that case, the light emerging out of the liquid crystal wavelength tunable filter 32 is maintained in the same direction of polarization as the incident light.

[Process Flow of Spectral Fundus Measurement]

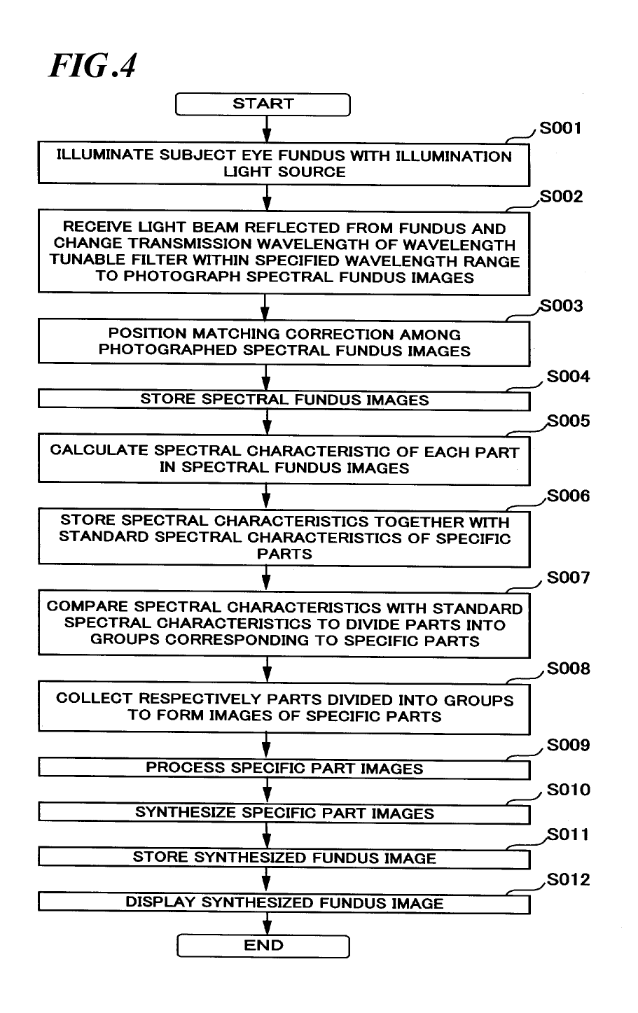

FIG. 4 shows an example process flow of a spectroscopic fundus measuring method of this embodiment. First, the fundus of a subject eye is illuminated with the illumination light source 11 (S001: subject eye illuminating step). The photographed image taking section 71 receives the illumination light beam reflected from the fundus and changes the transmission wavelength of the wavelength tunable filter 32 within a specified wavelength range to acquire photographed images of the spectral fundus image (S002: photographing step). The photographed images are acquired in succession from, for example, the short wavelength side. Next, the position correcting section 72 corrects the series of photographed spectral fundus images to match the positions of the same parts therein (S003: position correcting step). The series of corrected spectral fundus images are stored in the storage section 7A together with the series of photographed spectral fundus images (S004: first storing step). Next, the image extracting section 74 extracts spectral fundus images within wavelength ranges predetermined for respective specific parts from the series of corrected spectral fundus images (S005: image extracting step). The wavelength ranges may be determined by measuring the contrasts between the specific parts and their backgrounds or determined according to an empirical rule. The extracted images are displayed on the display section 7B (S006: first displaying step). Next, the image choosing section 75 chooses one of the extracted spectral fundus images or one of images of each of divided areas in the extracted spectral fundus images for each of the specific parts as the clearest image (S007: image choosing step). FIG. 4 shows an example in which one of images of each of divided areas is chosen. Next, in the case where the image choosing section 75 chooses one of images of each of divided areas as the clearest image, the image connecting section 76 connects the clearest images of all the areas to form the clearest images of the entire fundus (S008: image connecting step). Next, the processing section 77 performs processing such as noise removal on the clearest images for respective specific parts chosen for the entire fundus image in the image choosing section 75 or the clearest images of the entire fundus for respective specific parts formed in the image connecting section 76 (S009: processing step). Next, the image synthesizing section 78 synthesizes the fundus images processed for respective specific parts to form a high-contrast synthesized fundus image (S010: image synthesizing step). The formed high-contrast synthesized fundus image is stored in the storage section 7A (S011: second storing step), and displayed on the display section 7B (S012: second displaying step).

[Position Correction (Registration)]

The subject of measurement is a patient in a medical site, and it is preferred that the patient can be subjected to the measurement as comfortably as possible. In this embodiment, the measurement can be made without irradiating the fundus of a patient with strong light of a flash although the measurement takes a little longer time than the measurement with an ordinary fundus camera. However, there is a possibility that the fundus image is shifted to some extent by the influence of eye motion and so on within 20 seconds, in spite of comfortableness for a patient. The present inventors therefore developed a technique for matching the positions of a series of spectral fundus images of different wavelengths (registration technique). With it, spectroscopic analysis can be conducted even when the alignment state between an eye and the fundus camera is changed (see Japanese Patent Application 2004-352093).

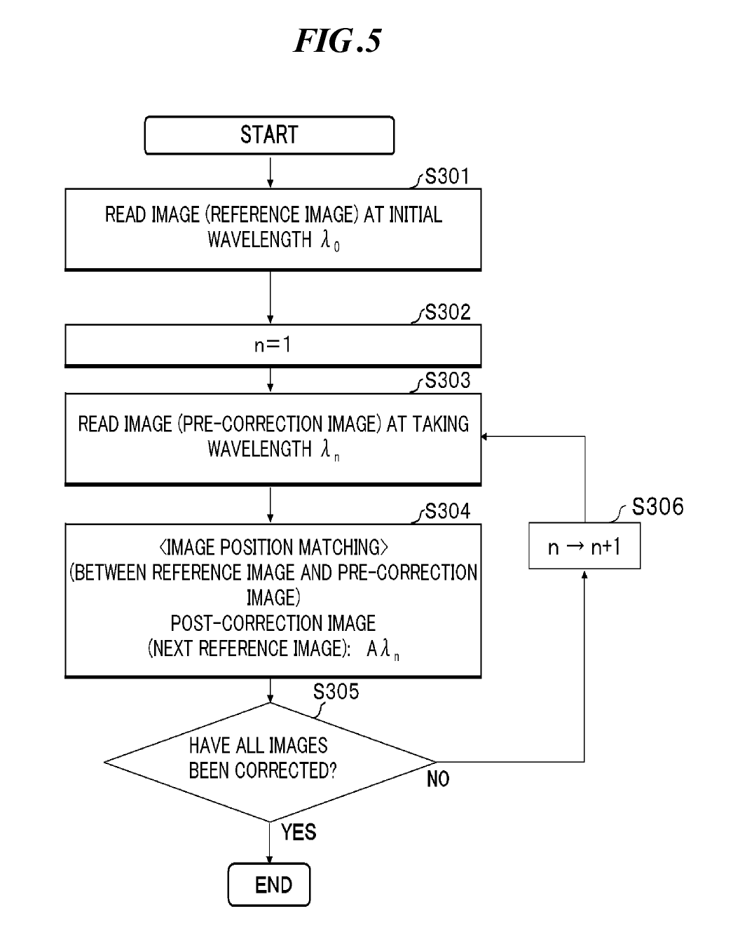

FIG. 5 shows an example flow of matching spectral fundus image positions. This corresponds to the step S003 of FIG. 4. As for taking a series of spectral fundus images, photographing at 10 nm intervals from 500 nm to 720 nm currently takes about 20 seconds under conditions of wavelength tuning time of the liquid crystal wavelength tunable filter 32, the exposure time of the CCD camera 34, etc. During that time, in many cases, undesirable displacements occur in alignment between the subject eye E and the fundus camera section 2 and in stationary viewing. As a result, the position of the spectral fundus images taken is displaced, and a position on the fundus images corresponding to the same coordinates on the light receiving surface of the CCD camera 34 is displaced. Therefore, the position correction have to be performed. The correction is made by position matching among a series of spectral fundus images. Besides, the spectral fundus image changes with the change in the wavelength, and the change in the spectral image is recognizable even at a glance when the wavelength change is large. As a result, images, taken at wavelengths apart from each other, of the same part on the fundus, are hard to interrelate. Therefore in this embodiment, alignment is corrected with reduced error as follows: First, position matching is made between two images taken at the shortest and second shortest wavelengths. Next, position matching is made between the images taken at the second shortest and the third shortest wavelengths, like a chain reaction. This image position matching is made in the position correcting section 72 of the image processing section 7.

First, a photographed fundus image is read at an initial (shortest) wavelength λ₀ to start taking images, and the image read is assumed to be a reference image (step S301). Next, the number (n) of times of image position matching is set to one (step S302). A photographed fundus image as an object of position matching (next shortest in wavelength to the reference image, called an image at a taking wavelength λₙ) is read, and the image is assumed to be a pre-correction image (step S303). Then, position matching is done between the reference image and the pre-correction image to correct its position. The pre-correction image with its position corrected is now assumed to be a new reference image (step S304). If any image not corrected remains (NO in the step S305), n is incrementally increased (step S306), a fundus image photographed at the next taking wavelength λₙ is read (step S303). The image position matching is repeated until the correction is made to all the photographed fundus images (YES in the step S305). Incidentally, reading the photographed fundus images in this flow may be re-reading the images already read into the storage section 7A by the image processing section 7 from the CCD camera 34 via the photographed image taking section 71, into the position correcting section 72. The flow of spectral fundus image position matching, including the loop process, may be controlled by a program. The program is stored in the image processing section 7, and the image position matching is carried out in the position correcting section 72.

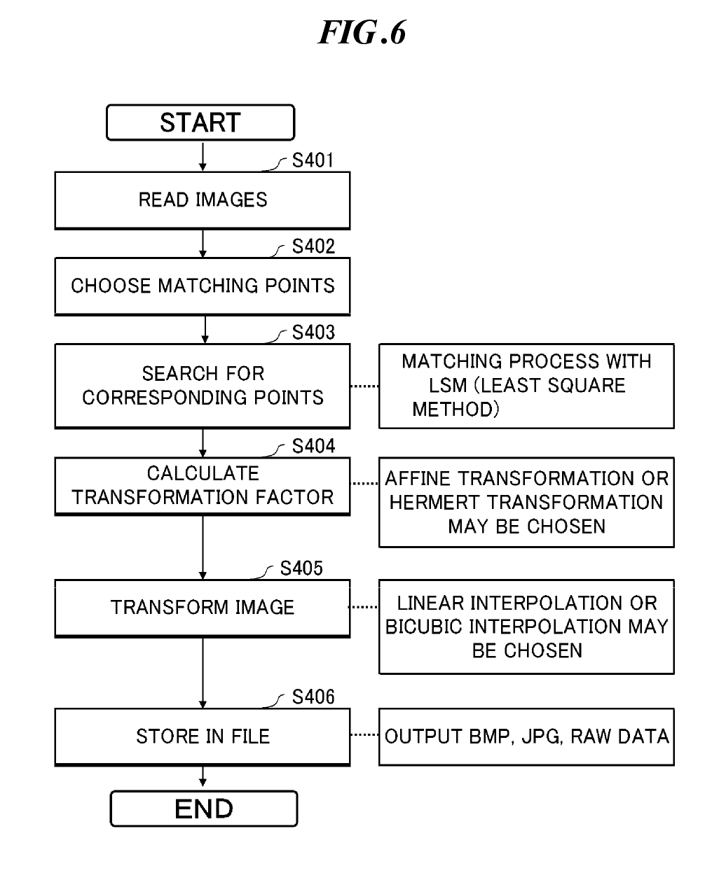

FIG. 6 shows an example flow of image position matching. It corresponds mostly to the step S304 of FIG. 5. Two spectral fundus images (reference image and pre-correction image (image taken at the adjacent wavelength)) of the illuminated fundus taken at different time points according to signals from the light receiving surface of the photographing section 4 are read (step S401) (This corresponds to the steps S301-S303 of FIG. 5. Steps S402 and after correspond to the step S304 of FIG. 5). Next, a plural number of characteristic points (points that are characteristic and highly conspicuous, may be linear in some cases) are chosen as image matching points from the two images (step S402). Next, positions of corresponding matching points are searched (step S403). For the search, for example the least squares matching (LSM) is used.

The least square matching is a method for performing the matching (to establish correlation) in which the position and shape of a template are fixed, and the position and shape of a matching window are changed so that the sum of the squares of the difference in shade becomes a minimum between the matching window and the template. For changing the position and shape of the matching window, the affine transformation or Hermert transformation may be chosen. As for these, difference in shade is calculated with varied transformation factors to determine the optimum factor (step S404). Next, transformation of the pre-correction image is carried out using the determined transformation factor (step S405). Here, a linear interpolation method or bicubic interpolation method may be chosen.

The bicubic method is a method for interpolating images and is called cubic interpolation method. As for the scanner in general, many models perform calculation with the primary interpolation method (calculation is made in reference to pixels on a straight line passing two points) or the nearest neighbor method. With the bicubic method, loss of information is the least, and in case of photographic images, the images obtained are smooth and natural. However, it takes much time because of complicated numerical operations. In contrast to the nearest neighbor method in which the value is determined from a single pixel in the neighborhood, the linear interpolation method determines the value from four pixels in the nearest neighborhood, so that interpolation accuracy is high in comparison with the nearest neighbor method.

Next, the image transformed from the pre-correction image is stored in a file (step S406). The stored image is used as a new reference image in the next image matching. The data may be stored for example in BMP format, in JPG format, or may be output as raw data.

[Image Analysis]

The images subjected to position matching by means of registration can be spectroscopically compared with one another. In this embodiment, the spectral characteristic of each part can be analyzed based on a series of spectral fundus images to determine wavelength ranges in which respective specific parts are clearly visible and to clarify whether each part belongs to any of the specific parts. In addition, the contrast between the brightness of each part and its background is calculated based on the series of spectral fundus images to contribute to the choice of images with maximum contrast.

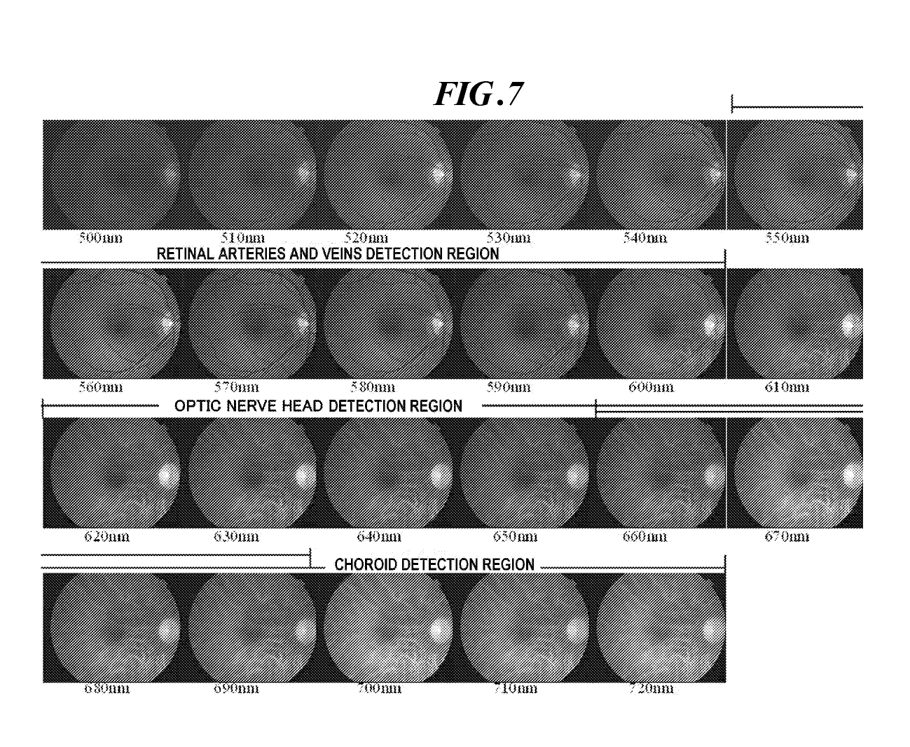

When the images subjected to registration are arranged in the order of wavelength as shown in FIG. 7 and a pixel within the image plane is designated, the changes of reflected light at each wavelength can be known. The spectral characteristics based on the series of spectral fundus images of different wavelengths include information on the structural and physical features of each part of the fundus.

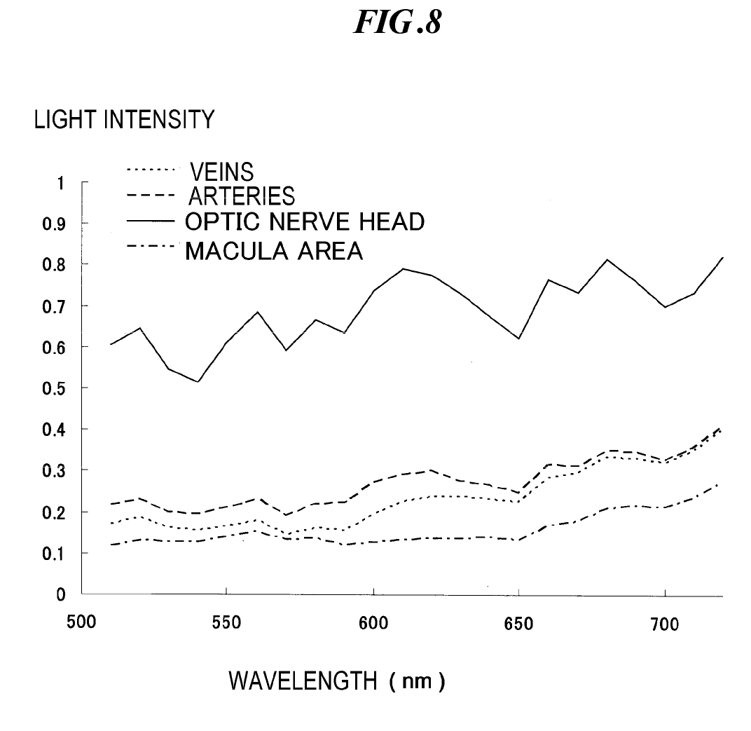

FIG. 8 shows an example of spectral characteristics of respective specific parts. The horizontal axis represents wavelength (nm) and the vertical axis represents received light intensity. The drawing shows the spectral characteristics of optic nerve head, retinal arteries, retinal veins and macula area. Optic nerve head exhibits by far the highest received light intensity among them, and retinal arteries, retinal veins and macula area exhibit relatively low received light intensities, decreasing in this order. In the reflection from the fundus, only the retina can be observed at a wavelength shorter than approximately 600 nm. At a wavelength longer than 600 nm, reflection from the choroid can be also observed. For example, when the fundus is photographed at 630 nm or 780 nm, an image of choroidal vessels can be obtained. Also, since the same part exhibits a similar spectral characteristic as shown in FIG. 8, each specific part can be identified based on the difference in spectral characteristic. In addition, the wavelength range in which a clear image can be obtained is different for each specific part. In other words, when an image is observed at a suitably chosen wavelength, the blood vessels in the choroid and the blood vessels in the retina, for example, can be distinguished.

[Extraction and Choice of Image]

FIG. 7 shows an example of hyperspectral image, that is, a sequence of spectral fundus images of different wavelengths. The images are photographed at 10 nm wavelength intervals in the wavelength range from 500 nm to 720 nm. The retinal arteries and veins are visible relatively clearly in the wavelength range from 550 nm to 600 nm, and this range is called retinal arteries and veins detection region. The optic nerve head is visible relatively clearly in the wavelength range from 620 nm to 690 nm, and this range is called optic nerve head detection region. The choroid is visible relatively clearly in the wavelength range from 660 nm to 720 nm, and this range is called choroid detection region. These regions, in which clear images of respective specific parts can be obtained, may be determined by measuring the contrast or may be determined empirically by the visual sense. Therefore, when the retinal arteries and veins need to be extracted, images in the wavelength range from 550 nm to 600 nm may be displayed, to choose therefrom the clearest image, that is, the highest contrast image, for example, an image of a wavelength of 570 nm, and to extract high-contrast parts as retinal arteries and veins. When the optic nerve head needs to be extracted, images in the wavelength range from 620 nm to 690 nm may be displayed, to choose therefrom the clearest image, that is, the highest contrast image, for example, an image of a wavelength of 640 nm, and to extract a high-contrast part as the optic nerve head. When the choroid needs to be extracted, images in the wavelength range from 660 nm to 720 nm may be displayed, to choose therefrom the clearest image, that is, the highest contrast image, for example, an image of a wavelength of 700 nm, and to extract a high-contrast part as the choroid. When the highest contrast image is chosen, the spectral fundus image region may be divided into a multiplicity of square areas and the highest contrast image may be chosen from the images of each area. In this embodiment, the highest contrast image is chosen from the images of each area. As described above, the specific parts such as retinal arteries and veins, optic nerve head and choroid can be extracted using spectral images.

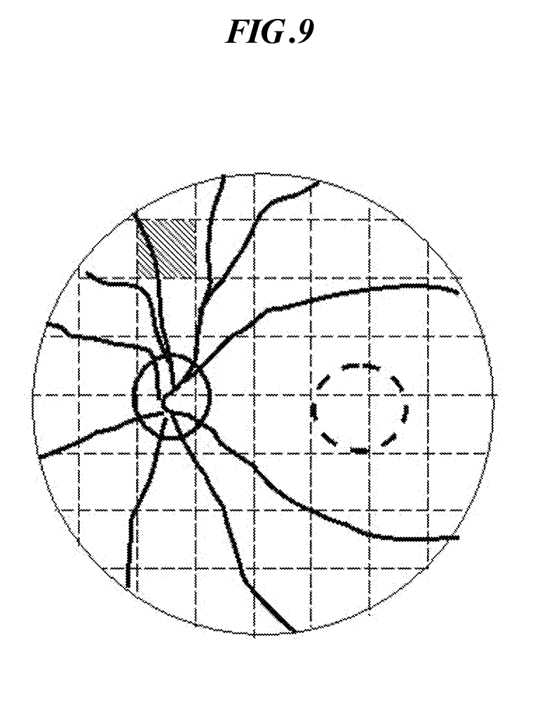

FIG. 9 shows an example of specific parts, here, an example of extracted image of retinal arteries and veins and optic nerve head. In the drawing, the very bright elliptical area on the left side of the center corresponds to the optic nerve head, and the thin linear parts extending radially therefrom correspond to the retinal arteries and veins. First, each spectral fundus image is divided into square areas, and the contrast in each area is calculated at each wavelength:
(Iₘₐₓ−Iₘᵢₙ)/(Iₘₐₓ+Iₘᵢₙ₎
(Iₘₐₓ: maximum value of brightness, Iₘᵢₙ: minimum value of brightness)

Then, the image with the highest contrast in the wavelength range for each of the objects (retinal arteries and veins, optic nerve head, choroid, etc.) is obtained from the images of each area and the chosen images are connected. That is, the image with the highest contrast is chosen from the images of each area in the images extracted from the retinal arteries and veins detection region ranging from 550 nm to 600 nm in the case of the retinal arteries and veins, from the optic nerve head detection region ranging from 620 nm to 690 nm in the case of the optic nerve head, from the choroid detection region ranging from 660 nm to 720 nm in the case of the choroid, and the chosen images are connected. Here, the contrast in the entire area is obtained to choose the clearest image. However, the present invention is not limited to the above example. When a specific part is distributed in specific regions within an area and the brightness is different at different points, the maximum value of the brightness of the specific part may be used to choose the image with the highest contrast as the clearest image for the specific part, or the maximum value of the differences in brightness between the specific part and the regions around it may be used to choose the image with the highest contrast as the clearest image for the specific part.

Each part can be detected based on its characteristics. For example, the retinal arteries and veins are characterized by their linear shape and low brightness, the optic nerve head is characterized by its elliptical shape, and the choroidal vessels are characterized by their linear shape and high brightness, and they can be extracted based on these characteristics. It is preferred to detect them in the following order: the optic nerve head, which is easy to detect in terms of image processing, the retinal arteries and veins extending linearly from the optic nerve head, and the choroidal vessels. Next, the processing section 77 performs processing such as noise removal on the connected images.

[Processing]

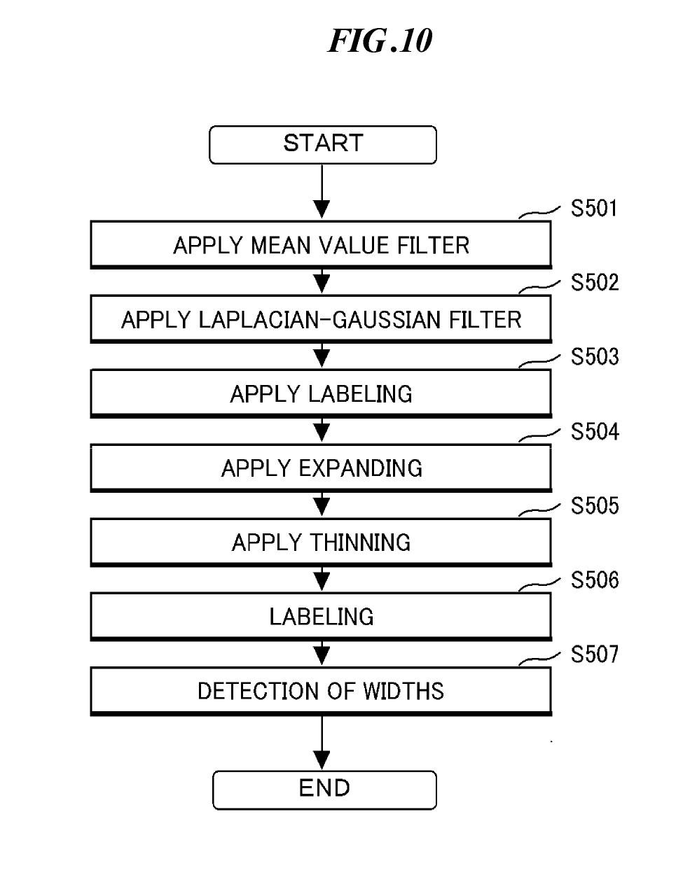

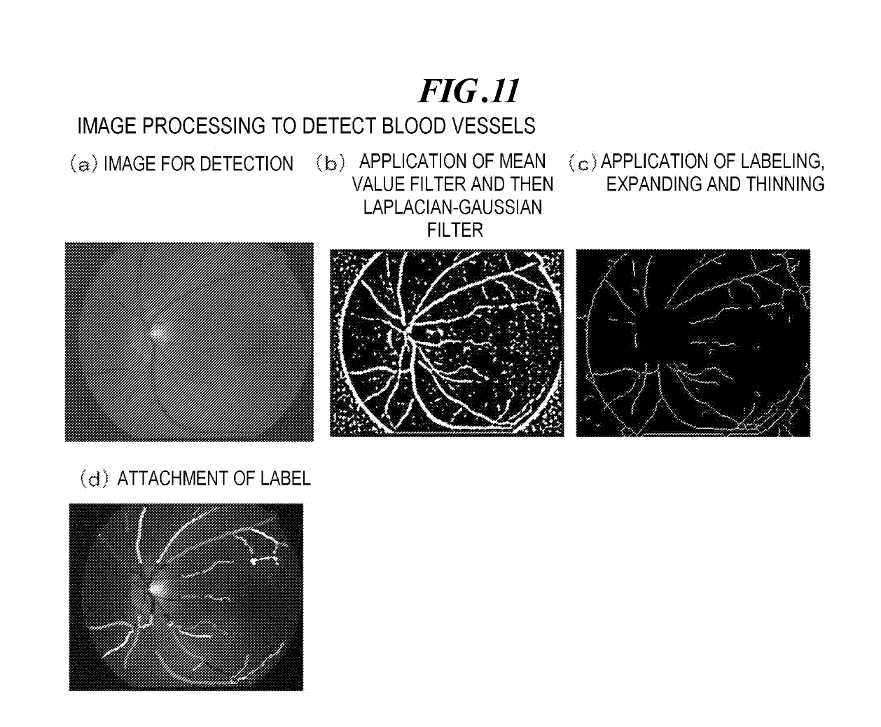

FIG. 10 shows the flow of processing, and FIG. 11 shows examples of images during the processing. The processing corresponds to S009 of FIG. 4. First, the connected image for each of specific parts is defined as an object image for detection (FIG. 11 (a)). Here, the retinal arteries and veins are the objects of detection. First, a mean value filter is applied (S501), and then a Laplacian-Gaussian filter is applied (S502) (FIG. 11 (b)). A mean value filter is an operator that averages pixels with their neighbors to remove noise, and a Laplacian-Gaussian filter is an operator that extracts edges with high contrast and applies a gaussian function to smooth them in order to remove noise. Next, labeling is carried out to give the same number to a series of parts with the same characteristic for identification (S503), expanding is carried out to expand points (S504), and thinning is carried out to reduce the width of lines in order to remove noise (S505) (FIG. 11 (c)). Next, a label is attached, that is, an attribute is attached to each of the numbered parts, and, here, the parts are displayed in color (S506) (FIG. 11 (d)). Next, the widths of the retinal arteries and veins are measured (S507). The widths are used as an index of health or medical condition. The processing is performed on the connected images for respective specific parts as described above to form clear fundus images without noise.

Next, the process returns to FIG. 4, and the image synthesizing section 78 synthesizes the fundus images processed for the chosen specific parts to form a high-contrast fundus image (S010). Colors are applied corresponding to the specific parts in the high-contrast fundus image, and the high-contrast fundus image is stored in the storage section 7A (S011). Then, the high-contrast fundus image is displayed in color on the display section 7B (S012).

As described above, according to this embodiment, there can be provided a spectroscopic fundus measuring apparatus capable of identifying each part in spectral fundus images based on its spectral characteristic easily and accurately and a measuring method therefor.

### Second Embodiment

An example in which each fundus image is divided into a plurality of areas, the clearest image with the highest contrast is chosen from images of each area in a plurality of extracted images, and the chosen images are connected to form the clearest images of the entire fundus is described in the first embodiment. In the second embodiment, an example in which the clearest image is manually chosen from images of each area in the extracted images is described. A plurality of extracted images are displayed on the display section 7B. When the operator designates a part in each of the areas, the spectral characteristic and the contrast of the part are displayed. The operator compares the spectral characteristic with the standard spectral characteristic of the specific part to confirm that the part belongs to the specific part, and chooses an extracted image with the highest contrast in a plurality of extracted images as the clearest image for the area. The clearest images chosen for all the areas are connected to form the clearest image of the entire fundus. The second embodiment is the same in other respects as the first embodiment and has the same effect as the first embodiment.

### Third Embodiment

An example in which the clearest image is manually chosen from the images of each area is described in the second embodiment. In the third embodiment, an example in which a plurality of extracted images are manually compared with one another to choose the clearest image of the entire fundus is described. Also in this case, when the operator designates a part in an extracted image, the spectral characteristic and contrast of the part are displayed. The operator compares the spectral characteristic with the standard spectral characteristic of a specific part to confirm that the part belongs to the specific part, and chooses an extracted image with the highest contrast from a plurality of extracted images as the clearest image. For example, an image of retinal arteries and veins is chosen from images of a wavelength of 570 nm, an image of optic nerve head from images of a wavelength of 640 nm, and an image of choroidal vessels from images of a wavelength of 700 nm as the clearest images, and the chosen images are synthesized. In this example, connection of images is not required. The specific parts are preferably displayed in different colors in the synthesized image so that each specific part can be distinguished at a glance. The third embodiment is the same in other respects as the first embodiment and has the same effect as the first embodiment.

### Fourth Embodiment

An example in which the clearest image is chosen from images of each area, and processing is performed after connecting the chosen images to remove noise is described in the first embodiment. In the fourth embodiment, an example in which processing is performed after the extraction of images and before choosing the clearest image. Although the processing is performed on one synthesized image in the first embodiment, the processing is performed on a plurality of extracted images in this embodiment. However, since calculation of contrasts is performed after the noise removal, the probability that the clearest image of each area can be properly chosen is higher. The fourth embodiment is the same in other respects as the first embodiment.

### Fifth Embodiment

An example in which one set of a series of spectral fundus images is taken is described in the first embodiment. In the fifth embodiment, two sets of a series of spectral fundus images are taken so that they can complement the position matching each other. That is, during 20 seconds of photographing, the spectral fundus image may be displaced. Thus, when the spectral fundus image is displaced relatively largely during photographing one set of images, the position matching is carried out among the other set of images with less displacement and the one set of images are converted to match them with the other set of images. The position matching can be thereby carried out reliably. The fifth embodiment is the same in other respects as the first embodiment.

### Sixth Embodiment

An example in which maximum and minimum values of brightness in each area are obtained to calculate the contrast therein in acquiring the contrast data is described in the first embodiment. In the sixth embodiment, an example in which the contrast between the brightness of each part and the brightness of its background is calculated is described. When a Laplacian-Gaussian filter is used, edge detection can be carried out to obtain the contrast between a part and its background. Therefore, instead of obtaining maximum and minimum values in each area, the contrasts between all the parts and their backgrounds can be obtained. This makes it possible to obtain more accurate contrast data. The sixth embodiment is the same in other respects as the first embodiment.

### Seventh Embodiment

Description is made without distinguishing the retinal arteries and the retinal veins in the first embodiment. In the seventh embodiment, an example in which the retinal arteries and the retinal veins are distinguished from each other is described.

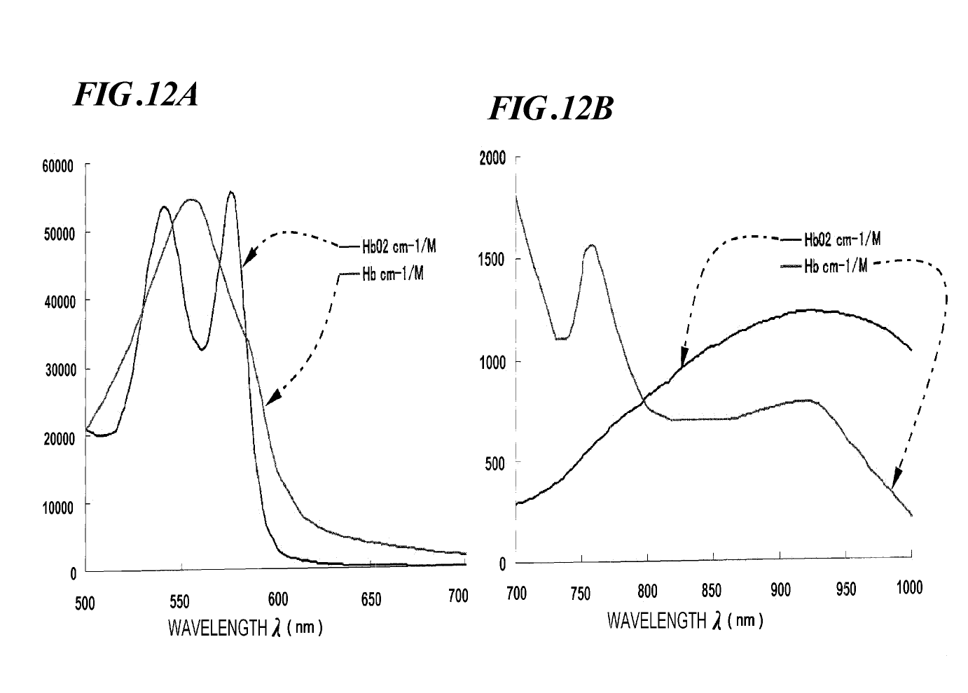

FIG. 12A and FIG. 12B show an example of absorbed light amounts of oxygenated hemoglobin and reduced hemoglobin (in cm⁻¹/moles/liter). FIG. 12A shows the absorbed light amount in the visible range and FIG. 12B in the near infrared range. Oxygenated hemoglobin is expressed as HbO₂ and the reduced hemoglobin as Hb. The oxygen saturation degree analysis for the retina utilizes the presence of wavelength-dependent difference in the amounts of absorbed light between oxygenated hemoglobin and reduced hemoglobin. Analyzing to what extent this spectral characteristic pattern is contained in the spectral characteristic of respective measurement subject parts makes it possible to determine the rates of content of oxygenated hemoglobin and reduced hemoglobin in the measurement subject parts. There is further a possibility of finding out the oxygen saturation degree from the rate of oxygenated hemoglobin. According to FIG. 12A and FIG. 12B, the retinal arteries have greater brightness than the retinal veins at a wavelength of around 580 nm, and the retinal veins have greater brightness than the retinal arteries at a wavelength of around 550 nm. Thus, when spectral fundus images of a wavelength of around 580 nm and spectral fundus images of a wavelength of around 550 nm in a series of spectral fundus images are compared, the parts with high brightness in the former can be determined as retinal arteries and the parts with high brightness in the latter can be determined as retinal veins.

The present invention can be implemented as a program that enables a computer to perform the image processing method described in the above embodiments. The program may be stored in a memory incorporated in the control section 8, may be stored in a storage device provided inside or outside the system, or may be downloaded through the Internet. The present invention can be also implemented as a storage device in which the program is stored.

While embodiments of the invention are described above, the invention is not limited to the above embodiments. Rather, it is apparent that the invention may be modified in various ways.

For example, while an example in which the extracted spectral fundus images are displayed on the display section was described in the above embodiments, the spectral fundus images are not necessarily displayed on the display section when the clearest images are automatically chosen as in the first embodiment. Further, it is also possible to change the order of steps in this embodiment. For example, the fundus images may be stored at a time after taking spectral fundus images at all the wavelengths in the spectral measurement wavelength range, or each fundus image may be stored immediately after taking the spectral fundus image at each wavelength. Further, image position matching may be carried out successively while reading spectral fundus images (pre-correction images) from the CCD camera, or position matching among photographed images may be carried out successively, after reading into the photographed image taking section all the spectral fundus images from the CCD camera, while re-reading into the position correcting section the spectral fundus images (pre-correction images) accumulated in the storage section.

Further, while an example was described in which the programs for the spectral fundus image taking flow and the CCD camera exposure time setting flow are stored in the control section, and the programs for the spectral retinal image position matching flow, the spectral retinal image analysis flow and so on are stored in the image processing section, the control section may hold all of these programs to control the entire spectral fundus image measuring apparatus including the image processing section, or the control section may read these programs from an external recording device or CD ROM to control the spectroscopic fundus measuring apparatus. The interval at which the spectral fundus images are taken is not limited to 10 nm, and the spectral fundus images may be taken at intervals of, for example, 2.5 nm or 25 nm.

This invention is used in measuring spectral fundus images.

### DESCRIPTION OF REFERENCE NUMERALS AND SYMBOLS

1: spectral fundus image measuring apparatus (spectroscopic fundus image measuring apparatus)

2: fundus camera section

3: top housing section

4: photographing section

5: relay optical section

6: camera relay section

7: image processing section

7A: storage section

7B: display section

8: control section

9: extended section

10: illumination optical system

11: illumination light source (halogen lamp)

12: condenser lens

13: spectral characteristic correcting filter

14: diaphragm

15: mirror

16: relay lens

20: light receiving optical system

21: iris diaphragm

22: focusing lens

23: image forming lens

24: mirror

25: switching mirror

31: dichroic mirror

32: liquid crystal wavelength tunable filter

33: image forming lens

34: CCD camera

40: common optical system

41: beam splitter

42: objective lens

50: alignment optical system

51: alignment light source

52: dichroic mirror

53: image forming lens

54: monitoring camera

60: finder optical system

71: photographed image taking section

72: position correcting section

73: image analyzing section

74: image extracting section

75: image choosing section

76: image connecting section

77: processing section

78: image synthesizing section

81: exposure control section

82: wavelength control section

E: subject eye

F: fundus

What is claimed is:

1. A spectroscopic fundus measuring apparatus, comprising:
an illumination optical system having an illumination light source for illuminating a fundus;
a light receiving optical system for receiving a wavelength-tunable light beam reflected from the illuminated fundus to photograph a series of spectral fundus images of different wavelengths;
an image processing section for processing the spectral fundus images; and
a storage section for storing the spectral fundus images,
the image processing section having a position correcting section for correcting the series of spectral fundus images photographed by the light receiving optical system to match the positions of the same parts therein, and an image extracting section for extracting spectral fundus images in wavelength ranges predetermined for respective specific parts from the series of spectral fundus images corrected in the position correcting section.

2. The spectroscopic fundus measuring apparatus as recited in claim 1,
wherein the image processing section has an image choosing section for choosing one of the extracted spectral fundus images or one of images of each of divided areas in the extracted spectral fundus images for each of the specific parts as the clearest image.

3. The spectroscopic fundus measuring apparatus as recited in claim 2, wherein the image choosing section chooses the image in which the contrast between the brightness of a specific part and the brightness of the background thereof is the highest from the extracted spectral fundus images as the clearest image for the specific part.

4. The spectroscopic fundus measuring apparatus as recited in claim 2, further comprising:
a display section for displaying the extracted spectral fundus images,
wherein the image choosing section chooses an image designated by an operator for a specific part from the spectral fundus images extracted and displayed on the display section as the clearest image-for the specific part.

5. The spectroscopic fundus measuring apparatus as recited in claim 2,
wherein, in the case where the image choosing section chooses one of images of each of divided areas as the clearest image, the image processing section has an image connecting section for connecting the clearest images of all the areas to form the clearest images of the entire fundus.

6. The spectroscopic fundus measuring apparatus as recited in claim 4,
wherein the image processing section has an image analyzing section for calculating a spectral characteristic of each part on the spectral fundus images based on the series of spectral fundus images corrected in the position correcting section,
wherein the storage section stores the spectral characteristics of the parts together with standard spectral characteristics of the specific parts, and
wherein, when the operator designates a part on the spectral fundus images, the display section displays the spectral characteristic of the designated part stored in the storage section.

7. The spectroscopic fundus measuring apparatus as recited in claim 4,
wherein the image analyzing section calculates the contrast between the brightness of each of the parts and the brightness of the background thereof based on the series of spectral fundus images corrected in the position correcting section,
wherein the storage section stores the contrasts of the parts, and
wherein, when the operator designates a part on the spectral fundus images, the display section displays the contrast of the designated part stored in the storage section.

8. The spectroscopic fundus measuring apparatus as recited in claim 5,
wherein the image processing section has an image synthesizing section for synthesizing the clearest images of the entire fundus chosen by the image choosing section or the clearest images of the entire fundus formed by the image connecting section to form a synthesized fundus image.

9. The spectroscopic fundus measuring apparatus as recited in claim 6,
wherein the image processing section compares spectral fundus images of a wavelength of around 580 nm and spectral fundus images of a wavelength of around 550 nm in the series of spectral fundus images and determines higher-brightness parts in the former than in the latter as retinal arteries and higher-brightness parts in the latter than in the former as retinal veins.

10. The spectroscopic fundus measuring apparatus as recited in claim 1,
wherein the wavelength of the illumination light beam from the illumination light source is tunable or the illumination optical system or the light receiving optical system has a wavelength selective filter.

11. A spectroscopic fundus measuring method, comprising:
a subject eye illuminating step of illuminating a fundus of a subject eye of an animal with a light beam from an illumination light source emitting a light beam in a specified wavelength range;
a photographing step of receiving a wavelength-tunable light beam reflected from the illuminated fundus to photograph a series of spectral images of the fundus of the animal of different wavelengths;
an image processing step of processing the spectral fundus images; and
a storing step of storing the spectral fundus images,
the image processing step having a position correcting step of correcting the series of spectral fundus images photographed in the photographing step to match the positions of the same parts therein, and an image extracting step of extracting spectral fundus images in wavelength ranges predetermined for respective specific parts from the series of spectral fundus images corrected in the position correcting step.

12. The spectroscopic fundus measuring method as recited in claim 11,
wherein the image processing step has an image choosing step of choosing one of the extracted spectral fundus images or one of images of each of divided areas in the extracted spectral fundus images for each of the specific part as the clearest image, and, in the case where one of images of each of divided areas is chosen as the clearest image in the image choosing step, an image connecting step of connecting the clearest images of all the areas to form the clearest images of the entire fundus.
